(19) United States
(12) Patent Application Publication
(10) Pub. No.: US 2011/0105931 A1
(43) Pub. Date: May 5, 2011

(54) System for Determining Patient Heart related Parameters for use in Heart Imaging

(75) Inventors: Wei Qu, Schaumburg, IL (US); Jinghua Chen, Schaumburg, IL (US); Yuanyuan Jia, Chicago, IL (US); Sukhveer Singh, Algonquin, IL (US); Michael J. Keller, Algonquin, IL (US)

(73) Assignee: Siemens Medical Solutions USA, Inc., Malvern, PA (US)

(21) Appl. No.: 12/247,630

(22) Filed: Oct. 8, 2008

Related U.S. Application Data
(60) Provisional application No. 60/989,215, filed on Nov. 20, 2007.

(51) Int. Cl. A61B 5/029; A61B 5/02

(52) U.S. Cl. 600/526; 600/508

(57) ABSTRACT

A system uses integrated spatio-temporal analysis in X-ray angiography, for example, by using spatial information within each image frame and temporal information between image frames to provide robust and accurate estimation of stroke area and volume, two and three dimensional ejection fraction and to accommodate patient heart variation. A system determines patient heart related parameters for use in patient heart imaging examination. An image data processor processes data representing multiple cardiac images of a patient over multiple heart beat cycles of the patient to derive data representing a distribution curve of a heart section area over multiple heart beat cycle times and indicating heart section area change over a heart beat cycle. An area processor determines a heart section area in response to user command. Also a computation processor determines a heart function parameter in response to the determined heart section area and the indicated heart section area change.

This is a non-provisional application of provisional application Ser. No. 60/989,215 filed Nov. 20, 2007, by W. Qu et al.

### FIELD OF THE INVENTION

This invention concerns a system for determining patient heart related parameters for use in patient heart imaging examination by determining a heart function parameter in response to a determined heart section area and a heart section area change.

### BACKGROUND OF THE INVENTION

X-Ray angiographic cardiac (e.g., left ventricular) analysis is used in a cardiac catheterization laboratory to assess and measure cardiac functions. In response to injection of a contrast medium into a left cardiac chamber, a left ventricular silhouette is viewed using a digital imaging system. The digital imaging system generates one or two image sequences (single plane or biplane) for further quantitative analysis. Further, an Ejection fraction (EF) is a cardiac function used routinely to judge if a patient has heart disease. It comprises,

$$EF = \frac{{V}_{ED} - {V}_{ES}}{{V}_{ED}} = \frac{SV}{{V}_{ED}} \qquad ( 1 )$$

where VED and VES are left ventricular volumes in end-diastolic (ED) phase and end-systolic (ES) phase, respectively.

Known systems extend angiocardiography to quantify a cardiac image for measurement of a left ventricular ejection fraction. Some known systems employ geometric assumptions of left ventricle shape and need to calculate three-dimensional left ventricular volumes for both end-diastolic (ED) and end-systolic (ES) left ventricle images. One known system determines a left ventricular ED value following intravenous injection of technetium. Another known system employs an “area-length” method using biplane angiocardiography for the measurement of left ventricular volume by processing projected areas and a long axis of the left ventricle to estimate left ventricle volume. A further known system extends the area-length method for single plane angiocardiograms by assuming that the left ventricular chamber can be represented by an ellipsoid of revolution (prolate spheroid) reference figure. An additional known system employs slice addition to calculate left ventricular volume.

The known systems are typically based on the analysis of static medical images and fail to consider temporal information inside an image sequence. Moreover, known systems employ a relatively strong assumption concerning left ventricle geometry without considering geometry difference between different patients. Furthermore, human interactions are needed to graphically select either a left ventricle contour or several ventricle control points for both ED and ES image frames for estimation of left ventricular volumes of ED and ES phases. This is burdensome, time consuming and sensitive to intra and inter observer errors. A system according to invention principles provides efficient and accurate determination of ejection fraction from two-dimensional image data for use in a wide range of clinical applications and addresses the identified deficiencies and related problems.

### SUMMARY OF THE INVENTION

A system uses integrated spatio-temporal analysis in X-ray angiography, for example, to automatically estimate stroke area and volume, two-dimensional ejection fraction and three-dimensional ejection fraction and to accommodate patient heart variation. A system determines patient heart related parameters for use in patient heart imaging examination. An image data processor processes data representing multiple cardiac images of a patient over multiple heart beat cycles of the patient to derive data representing a distribution curve of a heart section area over multiple heart beat cycle times and indicating heart section area change over a heart beat cycle. An area processor determines a heart section area in response to user command. Also a computation processor determines a heart function parameter in response to the determined heart section area and the indicated heart section area change.

### BRIEF DESCRIPTION OF THE DRAWING

FIG. 1 shows a system for determining patient heart related parameters for use in patient heart imaging examination, according to invention principles.

FIG. 2 shows a graphical model representation for use in X-ray left ventricle angiography, according to invention principles.

FIG. 3 illustrates a distribution curve of a heart section area over multiple heart beat cycle times and indicating heart section area change over a heart beat cycle, according to invention principles.

FIG. 4 illustrates long and short axes of a left ventricle as used by a system according to invention principles.

FIG. 5 shows a flowchart of a process performed by a system for determining patient heart related parameters for use in patient heart imaging examination, according to invention principles.

### DETAILED DESCRIPTION OF THE INVENTION

A system automatically estimates stroke area and volume, two-dimensional ejection fraction and three-dimensional ejection fraction using integrated spatio-temporal analysis and a geometric left ventricle model. The system advantageously uses spatial information within each image frame and temporal information between image frames to provide robust and accurate parameter estimation for use in X-ray angiography, for example. The system exploits the temporal correlation between end-diastolic (ED) and end-systolic (ES) phases, to advantageously eliminate a need to calculate left ventricle volumes for both ED and ES phases. Instead, the system automatically estimates a left ventricle stroke area and derives the relationship between two-dimensional ejection fraction and three-dimensional ejection fraction. The system applies a constraint instead of a known geometric left ventricular model and is readily adapted to accommodate patient heart variation and minimizes need for human interaction to achieve robust and accurate performance relative to known systems.

A processor as used herein is a device for executing stored machine-readable instructions for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A user interface (UI), as used herein, comprises one or more display images, generated by a user interface processor and enabling user interaction with a processor or other device and associated data acquisition and processing functions.

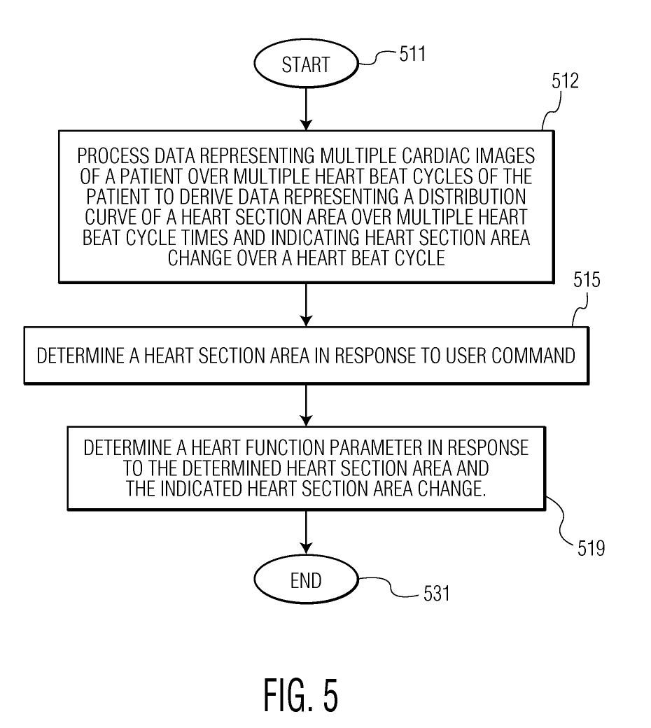

The UI also includes an executable procedure or executable application. The executable procedure or executable application conditions the user interface processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps (e.g., of FIG. 5) herein may be performed automatically or wholly or partially in response to user command. An activity (including a step) performed automatically is performed in response to executable instruction or device operation without user direct initiation of the activity. An object or data object comprises a grouping of data, executable instructions or a combination of both or an executable procedure.

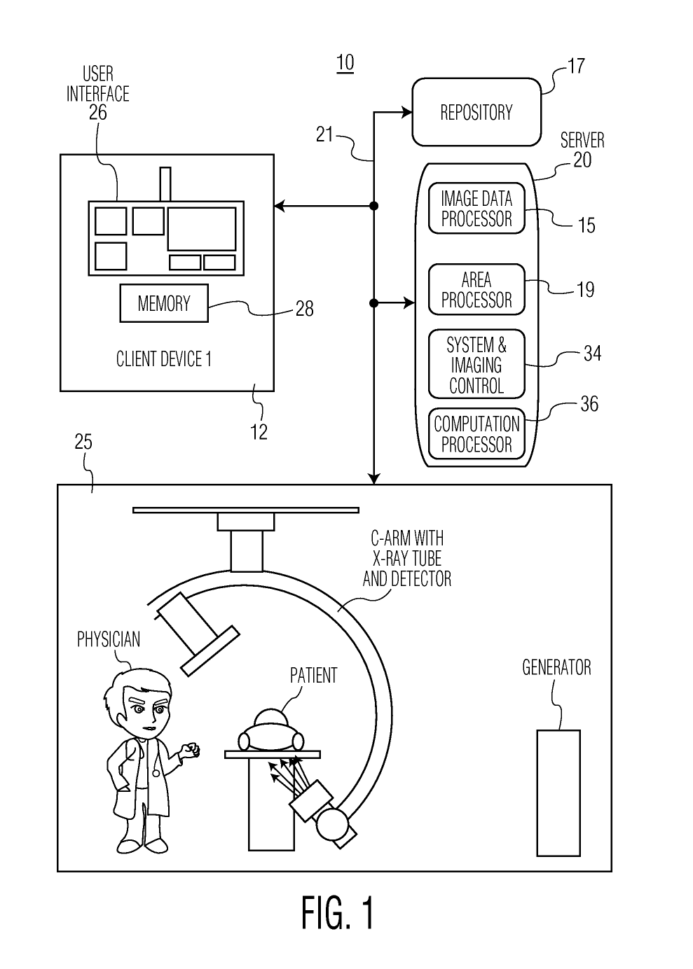

FIG. 1 shows system 10 for determining patient heart related parameters for use in patient heart imaging examination. System 10 includes one or more processing devices (e.g., workstation or portable device such as notebooks, Personal Digital Assistants, phones) 12 that individually include memory 28 and a user interface 26 supporting image presentation in response to user command and predetermined user (e.g., physician) specific preferences. System 10 also includes at least one repository 17, X-ray imaging modality system 25 (which in an alternative embodiment may comprise an MR (magnetic resonance), CT scan, or Ultra-sound system, for example) and server 20 intercommunicating via network 21. User interface 26 provides data representing display images comprising a Graphical User Interface (GUI) for presentation on workstation 12. At least one repository 17 stores medical image studies for multiple patients in DICOM compatible (or other) data format. A medical image study individually includes multiple image series of a patient anatomical portion which in turn individually include multiple images. Server 20 includes image data processor 15, area processor 19, computation processor 36 and system and imaging controller 34.

Image data processor 15 processes data representing multiple cardiac images of a patient over multiple heart beat cycles of the patient to derive data representing a distribution curve of a heart section area over multiple heart beat cycle times and indicating heart section area change over a heart beat cycle. The distribution curve indicates an end-diastolic (ED) location and end-systolic (ES) location that may be user selected or automatically detected by one of a number of known methods. Area processor 19 determines a heart section area in response to user command. Computation processor 36 determines a heart function parameter in response to the determined heart section area and the indicated heart section area change. System 10 acquires data representing multiple temporally sequential individual images of a patient organ (e.g., a heart) using X-ray modality system 25. X-ray modality system 25 comprises a C-arm X-ray radiation source and detector device rotating about a patient table and an associated electrical generator for providing electrical power for the X-ray radiation system.

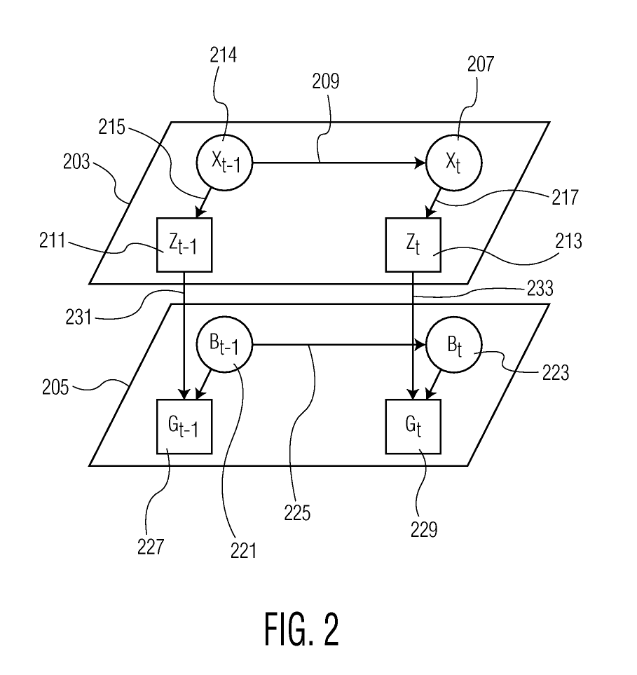

FIG. 2 shows a graphical model representation for use in X-ray left ventricle Spatio-Temporal Analysis for use in angiography, for example. Image data processor 15, in one embodiment automatically detects ED and ES image frames in a cardiac image sequence using one of a variety of known methods. In another embodiment the ED and ES image frames are determined by image data processor 15 in response to user command.

An X-ray left ventricular angiogram is formulated by a dynamic graphical model as illustrated in FIG. 2 and employed by image data processor 15 (FIG. 1). A first layer 203 represents the foreground data in an individual image frames and second layer 205 represents the background data in an individual image frame. Foreground state xt 207 is the area of a left ventricular region; t is the time index. The directed link 209 between consecutive states xt 207 and xt-1 214 represents a state transition density comprising a Markov chain. The square nodes 211, 213 in the foreground represent an observation z associated with each corresponding left ventricular state x. The directed links 215, 217 from x to its corresponding observation z represents a “generative” relationship and is characterized by a local observation likelihood p(z|x). In background layer 205, circle nodes 221, 223 represent background chest states B at each corresponding time. The background is not static but slightly changing due to chest (e.g., respiratory) movement, therefore directed link 225 between two consecutive background state nodes represent dynamic data change. Similar to the foreground state, the background chest state Bt 223 and Bt-1 221 also “generate” observations Gt 229 and Gt-1 227, in background layer 205. In an individual image frame, a foreground observation overlaps with a background observation. Links 231, 233 between foreground observation nodes and background observation nodes in an individual image frame reflects such kind of correlation.

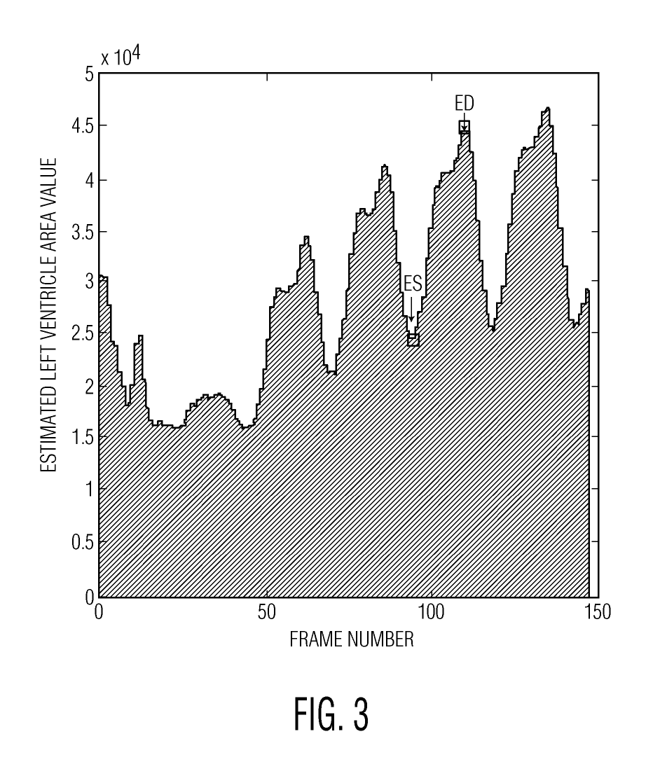

The FIG. 2 graphical model employed by image data processor 15 exploits the spatial image information within an individual image frame and also models the temporal correlation between consecutive image frames. Bayes rule applied to the graphical model advantageously provides a probability density propagation rule (a density updating equation)

$$p ( {x}_{1 ∷ t} \vert {z}_{1 ∷ t} , {G}_{1 ∷ t} ) = \frac{p ( {z}_{t} , {G}_{t} \vert {x}_{t} ) p ( {x}_{t} \vert {x}_{t - 1} )}{p ( {z}_{t} , {G}_{t} \vert {z}_{1 ∷ t - 1} , {G}_{1 ∷ t - 1} )} p ( {x}_{1 ∷ t - 1} \vert {z}_{1 ∷ t - 1} , {G}_{1 ∷ t - 1} ) \qquad ( 2 )$$

where the denominator is a constant since it is unrelated to the state xt; p(xt|xt-1) and the state transition density; p(x1:t-1|z1:t-1;G1:t-1) is the posterior in the previous step. The density p(zt,Gt|xt) is termed “background model-based likelihood” since it exploits background information and is modeled by an adaptive background process (model). Image data processor 15 estimates variation of left ventricle projected area in an angiographic image sequence using density updating equation (2) as exemplified in FIG. 3.

FIG. 3 illustrates a distribution curve of a heart section area over multiple heart beat cycle times and indicating heart section area change over individual heart beat cycles. In FIG. 3, the x-axis represents the image frame number. The y-axis represents the estimated left ventricular area in terms of number of pixels. The beginning part of the distribution curve is irregular due to background noise and/or X-ray detector movement before a contrast agent (dye) is injected into the left ventricle. After angiography begins, the curve becomes relatively regular with multiple cardiac cycles. The peak of an individual cycle corresponds to the ED phase while the valley is the ES phase. The estimated value of each peak [symbol] is not equal to the actual left ventricle projected area value in ED phase S_{ED} but contains some background noise Se1

S_{ED}=S_{ED}+Se1 (3)

Similarly, for ES phase

S_{ES}=S_{ES}+Se2 (4)

Since the image background movement has been adaptively modeled and determined to be substantially less than the left ventricle movement, it is further determined that background errors are similar throughout an image sequence after a contrast agent is injected into the left ventricle. Therefore,

Se1≈Se2 (5)

Further, image data processor 15 automatically calculates stroke area (SA) using equation (8) as follows,

$$\begin{matrix}SA = {S}_{\tilde{ED}} - {S}_{\tilde{ES}} & ( 6 ) \\ = ( {S}_{ED} - {S}_{ES} ) + ( {S}_{{e}_{1}} - {S}_{{e}_{2}} ) & ( 7 ) \\ = {S}_{ED} - {S}_{ES} , & ( 8 )\end{matrix}$$

where (sₑ₁−sₑ₂≈0<<(S_{ED}−S_{ES}). Since both S [symbol] and [symbol] are known from the estimated left ventricular area variation curve.

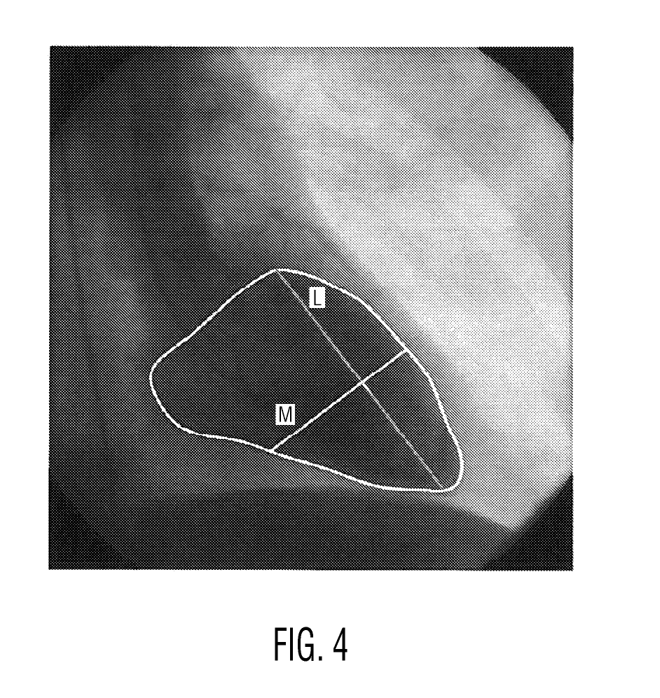

FIG. 4 illustrates long apex-to-aortic axis L and short axis M of a left ventricle as used by system 10. In automatically determining ejection fraction, image data processor 15 recovers three-dimensional volume information from two-dimensional projected left ventricular area (stroke area) in images and employs a geometric function. In a known area-length method, an ellipsoid model of the left ventricle is assumed. Further, a three-dimensional left ventricular volume is calculated by the formula

$$V = \frac{π}{6} {M}^{2} L . \qquad ( 9 )$$

where L is the long apex-to-aortic axis, and M is the short axis as shown in FIG. 4. M equals the length of the axis perpendicular to and bisecting the long apex-to-aortic axis of the ventricle. Also it is assumed

V=μ₁M²L. (10)

where μ₁ is a constant for a single patient but varies between different patients, which thus makes the left ventricle model adaptive to different people. Image data processor 15 employs the assumption that the left ventricular area is proportional to the product of M and L.

S=μ₂ML. (11)

where μ₂ is also a constant for a single patient but varies between different patients. Further, M_{ES}=αM_{ED} and L_{ES}=βL_{ED}, and EF_{3D} is the ejection fraction in terms of three-dimensional volume; EF_{2D} as the ejection fraction in terms of two-dimensional area. So,

$$\begin{matrix}{EF}_{2 D} = \frac{{S}_{ED} - {S}_{ES}}{{S}_{ED}} \approx \frac{{S}_{\tilde{ED}} - {S}_{\tilde{ES}}}{{S}_{ED}} & ( 12 ) \\ = \frac{{M}_{ED} {L}_{ED} - α {M}_{ED} β {L}_{ED}}{{M}_{ED} {L}_{ED}} & ( 13 ) \\ = 1 - αβ . & ( 14 )\end{matrix}$$

using equation (8) in (12). Similarly,

$$\begin{matrix}{EF}_{3 D} = \frac{{V}_{ED} - {V}_{ES}}{{V}_{ED}} & ( 15 ) \\ = \frac{{M}_{ED}^{2} {L}_{ED} - {α}^{2} {M}_{ED}^{2} β {L}_{ED}}{{M}_{ED}^{2} {L}_{ED}} & ( 16 ) \\ = 1 - {α}^{2} β . & ( 17 )\end{matrix}$$

By substituting equation (14) into (17),

EF_{3D}=1−α(1−EF_{2D})=1−α+αEF_{2D} (18)

Equation (18) gives a relationship between the two-dimensional left ventricular ejection fraction and the three-dimensional left ventricular ejection fraction. As long as parameter α and the area value S_{ED} are known, the three-dimensional ejection fraction is calculated by image data processor 15.

Image data processor 15 advantageously improves left ventricular ejection fraction measurement accuracy. The area-length formula is only a special case where

$${μ}_{1} = \frac{π}{6} .$$

The left ventricles of different people may vary even though the majority of patient left ventricles may be modeled as an ellipsoid. Assuming one model for different patients and neglecting variation between patients may lead to an erroneous result. Therefore image data processor 15 employs a geometric left ventricle model accommodating variation between different patients. Also the system advantageously requires that image graphical left ventricle segmentation needs to be performed once to estimate the area value S_{ED} in an ED image frame. Parameter α is estimated by selecting two more points in an ES image frame without doing segmentation. Since finding the short axis is much easier than detecting an entire left ventricle contour in an ES image frame.

In contrast to system 10 advantageously involving a single contour segmentation, known systems typically need to perform segmentation for both ED and ES image frames respectively. In known systems, accurate segmentation of the left ventricle in ED and/or ES frames is needed for accurate measurement of left ventricular ejection fraction. Further, such segmentation is burdensome and error prone due to the need for multiple user interactions to select either an initial contour or several control points. Consequently, segmentation results vary between different clinicians and/or at different performance times even using the same segmentation algorithm. System 10 increases measurement robustness and accuracy by minimizing user interaction and using a single contour segmentation. Further, because S_{ED}>S_{ES}, segmentation in an ED image frame is more robust and easier than in an ES image frame. In one embodiment S_{ED} in equation (18) is automatically estimated.

FIG. 5 shows a flowchart of a process performed by system 10 for determining patient heart related parameters for use in patient heart imaging examination. In step 512, following the start at step 511, image data processor 15 processes data representing multiple cardiac images of a patient over multiple heart beat cycles of the patient to derive data representing a distribution curve of a heart section (e.g., Left Ventricle or Right Ventricle) area over multiple heart beat cycle times and indicating heart section area change over a heart beat cycle. The distribution curve indicates an end-diastolic (ED) location and end-systolic (ES) location and image data processor 15 automatically determines the heart section area change from data comprising the distribution curve. Also the heart section area change comprises a change in area of the heart section indicated by the distribution curve between the ED and ES locations. In step 515, area processor 19 determines a heart section area in response to user command and selection of at least one of, (a) at least a portion of the heart section area contour and (b) control points of the heart section area.

Computation processor 36, in step 519, automatically determines a heart function parameter (e.g., heart ejection fraction, heart left ventricle volume or heart left ventricle stroke area) in response to a single determination of heart section area and the indicated heart section area change. The heart ejection fraction comprises at least one of, (a) a two dimensional heart ejection fraction and (b) a three dimensional heart ejection fraction. Further, computation processor 36 determines a three dimensional heart function parameter comprising a heart ejection fraction in response to a two dimensional ejection fraction value and values of a left ventricle long apex to aortic axis and short apex to aortic axis. Computation processor 36 determines a heart function parameter comprising a heart ejection fraction in response to a single determination of left ventricle area corresponding to at least one of, (a) an end-diastolic (ED) point and (b) an end-systolic (ES) point and combining a determined left ventricle area value and a left ventricle area change value. Computation processor 36 determines a heart left ventricle volume in response to values of a left ventricle long apex to aortic axis and short apex to aortic axis. The process of FIG. 5 terminates at step 531.

The systems and processes of FIGS. 1-5 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system automatically estimates stroke area and volume, two-dimensional ejection fraction and three-dimensional ejection fraction using integrated spatio-temporal analysis and a geometric left ventricle model and accommodates patient heart variation. The processes and applications may, in alternative embodiments, be located on one or more (e.g., distributed) processing devices accessing a network linking the elements of FIG. 1. Further, any of the functions and steps provided in FIGS. 1-5 may be implemented in hardware, software or a combination of both and may reside on one or more processing devices located at any location of a network linking the elements of FIG. 1 or another linked network, including the Internet.

What is claimed is:

1. A system for determining patient heart related parameters for use in patient heart imaging examination, comprising:
an image data processor for processing data representing a plurality of cardiac images of a patient over a plurality of heart beat cycles of said patient to derive data representing a distribution curve of a heart section area over a plurality of heart beat cycle times and indicating heart section area change over a heart beat cycle;
an area processor for determining a heart section area in response to user command; and
a computation processor for determining a heart function parameter in response to the determined heart section area and the indicated heart section area change.

2. A system according to claim 1, wherein
said distribution curve indicates an end-diastolic (ED) location and end-systolic (ES) location and
said heart section area change comprises a change in area of said heart section indicated by said distribution curve between said ED and ES locations and
said heart function parameter is determined in response to a single determination of heart section area.

3. A system according to claim 2, wherein
said image data processor automatically determines said heart section area change from data comprising said distribution curve.

4. A system according to claim 1, wherein
said area processor determines said heart section area in response to user command and selection of at least one of, (a) at least a portion of the heart section area contour and (b) control points of the heart section area.

5. A system according to claim 1, wherein
said computation processor determines a heart function parameter comprising a heart ejection fraction.

6. A system according to claim 5, wherein
said heart ejection fraction comprises at least one of, (a) a two dimensional heart ejection fraction and (b) a three dimensional heart ejection fraction.

7. A system according to claim 1, wherein
said computation processor determines a three dimensional heart function parameter comprising a heart ejection fraction in response to a two dimensional ejection fraction value and values of a left ventricle long apex to aortic axis and short apex to aortic axis.

8. A system according to claim 1, wherein
said heart section area comprises a left ventricle area and
said computation processor determines a heart function parameter comprising a heart ejection fraction in response to a single determination of left ventricle area corresponding to at least one of, (a) an end-diastolic (ED) point and (b) an end-systolic (ES) point.

9. A system according to claim 8, wherein
said computation processor determines said heart ejection fraction, in response to combining a determined left ventricle area value and a left ventricle area change value.

10. A system according to claim 1, wherein
said heart section comprises at least one of, (a) a Left Ventricle and (b) a Right Ventricle.

11. A system according to claim 1, wherein
said computation processor determines a heart function parameter comprising a heart left ventricle volume in response to values of a left ventricle long apex to aortic axis and short apex to aortic axis.

12. A system according to claim 1, wherein
said heart section area comprises a left ventricle area and
said computation processor determines a heart function parameter comprising a heart ejection fraction in response to combining a determined left ventricle area value and a left ventricle area change value.

13. A system according to claim 1, wherein
said computation processor automatically determines a heart function parameter comprising a heart left ventricle stroke area

14. A system for determining patient heart related parameters for use in patient heart imaging examination, comprising:
an image data processor for processing data representing a plurality of cardiac images of a patient over a plurality of heart beat cycles of said patient to derive data representing a distribution curve of a left ventricle area over a plurality of heart beat cycle times and indicating left ventricle area change over a heart beat cycle;
an area processor for determining left ventricle area in response to user command; and
a computation processor for automatically determining at least one of, (a) heart left ventricle volume and (b) a heart left ventricle stroke area, in response to the determined left ventricle area and the indicated left ventricle area change.

15. A system according to claim 14, wherein
said computation processor automatically determines a heart ejection fraction, in response to the determined left ventricle area and the indicated left ventricle area change.

16. A system according to claim 14, wherein
said heart ejection fraction comprises at least one of, (a) a two dimensional heart ejection fraction and (b) a three dimensional heart ejection fraction.

17. A system according to claim 14, wherein
said computation processor determines a three dimensional heart function parameter comprising a heart ejection fraction in response to a two dimensional ejection fraction value and values of a left ventricle long apex to aortic axis and short apex to aortic axis.

18. A system according to claim 14, wherein
said left ventricle also comprises a right ventricle.

19. A system according to claim 14, wherein
said computation processor determines said heart left ventricle volume in response to values of a left ventricle long apex to aortic axis and short apex to aortic axis.

20. A system for determining patient heart related parameters for use in patient heart imaging examination, comprising:
an image data processor for processing data representing a plurality of cardiac images of a patient over a plurality of heart beat cycles of said patient to derive data representing a distribution curve of a left ventricle area over a plurality of heart beat cycle times and indicating left ventricle area change over a heart beat cycle;
an area processor for determining left ventricle area in response to user command; and
a computation processor for determining a heart ejection fraction, in response to the determined left ventricle area and the indicated left ventricle area change.

21. A system according to claim 20, wherein
said computation processor determines a three dimensional heart ejection fraction in response to at least one of, (a) a value of a left ventricle long apex to aortic axis and (b) a left ventricle short apex to aortic axis.

22. A system according to claim 20, wherein
said heart ejection fraction is determined in response to a single determination of left ventricle area corresponding to at least one of, (a) an end-diastolic (ED) point and (b) an end-systolic (ES) point.

23. A system according to claim 20, wherein
said computation processor determines a heart ejection fraction, in response to combining a determined left ventricle area value and the indicated left ventricle area change value.
